(19) United States
(12) Patent Application Publication
(10) Pub. No.: US 2023/0357336 A1
(43) Pub. Date: Nov. 9, 2023

(54) PHOSVITIN COMPOSITIONS AND METHODS OF MAKINGAND USING

(71) Applicant: BET BIOSCIENCE EXTRACTION TECHNOLOGIES INC., Abbotsford (CA)

(72) Inventors: Willem Marinus TEN HAAF, Langley (CA); Arno Daniel NEUMANN, Abbotsford (CA)

(21) Appl. No.: 18/014,712

(22) PCT Filed: Jul. 7, 2021
(86) PCT No.: PCT/CA2021/050925

§ 371 Date: Jan. 5, 2023

Related U.S. Application Data
(60) Provisional application No. 63/048,781, filed on Jul. 7, 2020.

(51) Int. Cl. C07K 14/465; A23J 1/09; A23L 33/17

(52) U.S. Cl. CPC C07K 14/465; A23J 1/09; A23L 33/17

(57) ABSTRACT

Disclosed are novel compositions containing the egg yolk protein phosvitin or peptide fragments thereof. The compositions are substantially free of mineral ions chelated to the phosvitin protein or peptide fragments thereof. The compositions may be used in the formulation of products, including food products, personal care products, or pharmaceutical products, for example, to preserve the products.

### RELATED APPLICATION

This application claims the benefit of United States Provisional Patent Application No. 63/048,781, filed Jul. 7, 2020; the entire contents of Patent Application No. 63/048,781 are hereby incorporated by reference.

### FIELD OF THE DISCLOSURE

The present disclosure relates to poultry eggs and poultry egg proteins. In particular, the present disclosure relates to the egg yolk protein phosvitin.

### BACKGROUND OF THE DISCLOSURE

The following paragraphs are provided by way of background to the present disclosure. They are not, however, an admission that anything discussed therein is prior art or part of the knowledge of persons skilled in the art.

Poultry eggs are an important source of proteins and other nutrients and are therefore globally included in the human diet. A significant quantity of all produced poultry eggs are consumed by humans as food ingredients. In some instances, in baking applications, for example, whole eggs are used as food ingredients. However, it is frequently desirable to use only a portion of an egg as a food ingredient. Thus, for example, egg yolks are used in the manufacture of mayonnaise and various other foods.

The egg yolk constitutes approximately one third of the liquid weight of an egg, and is high in fats and fatty acids. Furthermore, egg yolk contains fat soluble vitamins (vitamin A, D, E, and K), as well as unsaturated fatty acids (e.g., oleic acid, linoleic acid, and linolenic acid) and saturated fatty acids (e.g., palmitic acid, stearic acid, and myristic acid). Egg yolks also contain proteins, typically from about 15.5% to about 16.5% of egg yolk in hen eggs. One such protein, phosvitin, is a phosphoprotein and represents about 11% of egg yolk protein. Biosynthetically, phosvitin is derived from a large multidomain protein, known as vittelogenin, which is synthesized in the liver of laying hens and serves as a precursor protein. Stimulated by estrogen, vitellogenin is cleaved into phosvitin, lipovitellin and other yolk proteins. Phosvitin has a molecular weight of 35 kDa and contains about 12% nitrogen and about 10% phosphorus. Furthermore, phosvitin contains 217 amino acid residues, of which 123 are serine residues. Of these 123 serine residues, 118 are phosphorylated, making phosvitin the most highly phosphorylated protein found in nature. Due to the large amount of negatively charged phosphoserine residues, phosvitin exhibits strong mineral chelating ability, and is believed to provide metal ions during embryonic development.

Several practical applications of phosvitin to promote human health are known to the art. For example, phosvitin has been suggested to be useful as a natural antioxidant and food preservative agent. However, under certain conditions phosvitin is an ineffective antioxidant. Thus, for example, phosvitin was found to be unable to limit hemin mediated oxidation, or copper (Cu²⁺) mediated oxidation reactions at pH 7.8 in a phospholipid emulsion system (Samaraweera, H. et al., 2011 J. Food Science 76 (7) 143-150).

Therefore, there currently remain practical limitations to the utility of phosvitin and there remains a need in the art for improved phosvitin compositions.

### SUMMARY OF THE DISCLOSURE

The following paragraphs are intended to introduce the reader to the more detailed description that follows and not to define or limit the claimed subject matter of the present disclosure.

In one broad aspect, the present disclosure relates to compositions containing phosvitin. The compositions are substantially free of mineral ions chelated thereto, and are surprisingly potent as a preservative agent in the preparation of products requiring a preservative, including but not limited to, food products, personal care products, and pharmaceutical products.

Accordingly, in one aspect, the present disclosure provides, in accordance with the teachings herein, in at least one embodiment, a composition comprising phosvitin or peptide fragments thereof, wherein the phosvitin or peptide fragments thereof are substantially free of mineral ions chelated thereto.

In at least one embodiment, in an aspect, the phosvitin or peptide fragments thereof can be substantially free of bivalent mineral ions chelated thereto.

In at least one embodiment, in an aspect, the phosvitin or peptide fragments thereof can be substantially free of bivalent mineral ions chelated thereto, the mineral ions selected from the group consisting of calcium ions (Ca²⁺), iron ions (Fe²⁺), zinc ions (Zn²⁺), and manganese ions (Mn²⁺).

In at least one embodiment, in an aspect, the phosvitin or peptide fragments thereof can comprise less than about 148 ppm calcium (Ca²⁺) ions chelated thereto.

In at least one embodiment, in an aspect, the phosvitin or peptide fragments thereof can comprise less than about 3.2 ppm iron ions (Fe²⁺) chelated thereto.

In at least one embodiment, in an aspect, the phosvitin or peptide fragments thereof can comprise less than about 148 ppm calcium (Ca²⁺) ions and less than about 3.2 ppm iron ions (Fe²⁺) chelated thereto.

In at least one embodiment, in an aspect, the phosvitin or peptide fragments thereof can comprise less than about 148 ppm calcium (Ca²⁺) ions and less than about 3.2 ppm metallic mineral ions chelated thereto.

In at least one embodiment, in an aspect, the metallic mineral ions can be iron ions (Fe²⁺), zinc ions (Zn²⁺), and manganese ions (Mn²⁺).

In at least one embodiment, in an aspect, the phosvitin or peptide fragments thereof can comprise less than about 148 ppm calcium ions (Ca²⁺), less than about 3.2 ppm iron ions (Fe²⁺), less than 2.3 ppm zinc ions (Zn²⁺), and less than about 0.033 ppm manganese ions (Mn²⁺) chelated thereto.

In at least one embodiment, in an aspect, the phosvitin or peptide fragments thereof, can comprise about 53.2 ppm or less calcium ions (Ca²⁺), about 2.6 ppm or less iron ions (Fe²⁺), about 2.2 ppm or less zinc ions (Zn²⁺), and about 0.018 ppm or less manganese ions (Mn²⁺) chelated thereto.

In at least one embodiment, in an aspect, the phosvitin or peptide fragments thereof can constitute at least about 95% (w/w), at least about 96% (w/w), at least about 97% (w/w), at least about 98% (w/w), or at least about 99% (w/w) of the composition.

In at least one embodiment, in an aspect, the phosvitin or peptide fragments thereof can constitute at least about 95% (w/w), at least about 96% (w/w), at least about 97% (w/w), at least about 98% (w/w), or at least 99% about (w/w) of the composition, and the composition comprises no more than about 148 ppm calcium ions (Ca²⁺), and no more than about 3.2 ppm metallic mineral ions chelated thereto.

In at least one embodiment, in an aspect, the composition further can comprise at least one of a diluent, carrier, or auxiliary agent.

In at least one embodiment, in an aspect, the diluent can be water.

In at least one embodiment, in an aspect, the phosvitin or peptide fragments thereof together with the diluent, carrier, or auxiliary agent, can constitute at least about 95% (w/w), at least about 96% (w/w), at least about 97% (w/w), at least about 98% (w/w), or at least about 99% (w/w) of the composition, and the composition comprises no more than about 148 ppm calcium ions (Ca²⁺), and no more than about 3.2 ppm metallic mineral ions chelated thereto.

In at least one embodiment, in an aspect, the metallic mineral ions can be iron ions (Fe²⁺), zinc ions (Zn²⁺) and manganese ions (Mn²⁺).

In at least one embodiment, in an aspect, the composition can exhibit antimicrobial properties.

In at least one embodiment, in an aspect, the composition can exhibit anti-oxidant properties.

In at least one embodiment, in an aspect, the phosvitin can be a protein comprising SEQ.ID NO: 1, or a sequence that is at least 80% identical thereto.

In at least one embodiment, in an aspect, the phosvitin peptide fragments can have a polypeptide sequence selected from the group consisting of SEQ.ID NO: 3; SEQ.ID NO: 4; SEQ.ID NO: 5; SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, or a sequence that is at least 80% identical thereto.

In at least one embodiment, in an aspect, the phosvitin or peptide fragments thereof can be substantially free from egg proteins naturally associated therewith.

In another aspect, the present disclosure relates to preservative formulations containing phosvitin. Accordingly, the present disclosure further provides, in at least one embodiment, a preservative formulation comprising a composition comprising phosvitin or peptide fragments substantially free of mineral ions chelated thereto, as disclosed herein.

In at least one embodiment, in an aspect, the auxiliary agent can be selected from the group consisting of an antimicrobial peptide, a chelating agent, an essential oil, a photochemically active agent, a cannabinoid, a terpene, a bitter acid, a polyphenol, and a vitamin, or mixtures thereof.

In another aspect, the present disclosure relates to products containing phosvitin. Accordingly, the present disclosure further provides, in at least one embodiment, a method for formulating a product comprising:
(i) providing a composition comprising phosvitin or peptide fragments thereof, wherein the phosvitin or peptide fragments thereof are substantially free of mineral ions chelated thereto, as disclosed herein; and
(ii) formulating an effective preservative amount of the composition with additional ingredients suitable for formulating the product to thereby form the product.

In at one embodiment, in an aspect, the product can be a food product, personal care product, or pharmaceutical product.

In another aspect, the present disclosure further provides, in at least one embodiment, a product comprising:
(i) An effective preservative amount of a composition comprising phosvitin or peptide fragments thereof, wherein the phosvitin or peptide fragments thereof are substantially free of mineral ions chelated thereto, as disclosed herein; and
(ii) additional ingredients suitable for formulating the product.

In at least one embodiment, in an aspect, the product can be a food product, personal care product, or pharmaceutical product.

In another aspect, the present disclosure relates to uses of phosvitin. Thus, in an aspect, the present disclosure provides, in at least one embodiment, a use of a composition comprising phosvitin or peptide fragments thereof, wherein the phosvitin or peptide fragments thereof are substantially free of mineral ions bound thereto, as disclosed herein, in the manufacture of a product.

In at least one embodiment, in an aspect, the product can be a food product, personal care product, or pharmaceutical product.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWING

The disclosure is in the hereinafter provided paragraphs described, by way of example, in relation to the attached figure. The figure provided herein is provided for a better understanding of the example embodiments and to show more clearly how the various embodiments may be carried into effect. The figure is not intended to limit the present disclosure.

FIG. 1 is an SDS polyacrylamide gel stained with Coomassie Blue showing a purified phosvitin protein preparation (lane 2) and molecular weight markers (lane 1). The molecular weight of the molecular weight markers is indicated on the left hand side.

The figure together with the following detailed description make apparent to those skilled in the art how the disclosure may be implemented in practice.

### DETAILED DESCRIPTION OF THE DISCLOSURE

Various compositions, methods or processes will be described below to provide an example of an embodiment of each claimed subject matter. No embodiment described below limits any claimed subject matter and any claimed subject matter may cover processes, compositions or methods that differ from those described below. The claimed subject matter is not limited to compositions, processes or methods having all of the features of any one composition, system or process described below or to features common to multiple or all of the compositions, systems or methods described below. It is possible that a composition, method or process described below is not an embodiment of any claimed subject matter. Any subject matter disclosed in a composition, method or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such as “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”. The term “and/or” is intended to represent an inclusive or. That is “X and/or Y” is intended to mean X or Y or both, for example. As a further example, X, Y, and/or Z is intended to mean X or Y or Z or any combination thereof.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore, any range of values described herein is intended to specifically include the limiting values of the range, and any intermediate value or sub-range within the given range, and all such intermediate values and sub-ranges are individually and specifically disclosed (e.g., a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### Terms and Definitions

The term “phosvitin”, as used herein, refers to the so named polypeptide occurring in the yolk of poultry eggs and having, in the case of chicken (Gallus gallus) phosvitin, an amino acid sequence as described by Byrne, M. et al., 1984, Biochemistry, 23, 4275-4279, and shown herein as SEQ.ID NO: 1, and further refers to any and all proteins comprising a sequence of amino acid residues which is (i) substantially identical to SEQ.ID NO: 1, or (ii) encoded by a nucleic acid sequence capable of hybridizing under at least moderately stringent conditions to any nucleic acid sequence encoding SEQ.ID NO: 1, but for the use of synonymous codons, including SEQ.ID NO: 2 set forth herein. Peptide fragments of phosvitin may be generated by chemical or proteolytic digestion, for example, digestion by trypsin. Phosvitin peptide fragments contain multiple serine residues and comprise generally at least 10, at least 25, at least 50, at least 75, at least 100, or at least 150 consecutive amino acid residues of SEQ.ID NO: 1, or of a sequence of amino acid residues that is (i) substantially identical to SEQ.ID NO: 1, or (ii) encoded by a nucleic acid sequence capable of hybridizing under at least moderately stringent conditions to any nucleic acid sequence encoding SEQ.ID NO: 1, but for the use of synonymous codons, including SEQ.ID NO: 2 set forth herein.

By the term “substantially identical” it is meant that two amino acid sequences preferably are at least 70% identical, and more preferably are at least 85% or 90% identical, and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Mol. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log10 [Na+])+0.41(% (G+C)−6001/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5×sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

With the term “substantially free of mineral ions”, as used herein in reference to a composition, it is meant that such composition contains a quantity of mineral ions less than 150 ppm.

The term “metallic mineral ions”, as used herein, refers to mineral ions of metallic elements, generally designated with reference to the Period Table of Elements and include, without limitation, iron ions (Fe²⁺), zinc ions (Zn²⁺) and manganese ions (Mn²⁺).

The term “effective preservative amount”, as used herein, refers to an amount that is sufficient to effect a desired preservative effect. Such effect can include the prevention of spoilage of products, or the extension of the shelf life of a product, for example. The effective amount can vary depending on the product or the conditions to which the product is exposed.

The term “substantially pure”, as used herein, as may be used interchangeably herein, describe a compound, e.g., a polypeptide which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides or antibodies, by chromatography, gel electrophoresis or HPLC analysis.

### General Implementation

As hereinbefore mentioned, the present disclosure relates to compositions comprising phosvitin. The compositions of the present disclosure may, for example, be used to formulate a product requiring a preservative, including but not limited to, a food product, personal care product, or pharmaceutical product. The phosvitin containing compositions of the present disclosure are exceptionally low in mineral ions chelated to phosvitin, notably the compositions of the present disclosure can contain mineral ions in amount of 150 ppm or less. Moreover, the phosvitin containing compositions exhibit a surprising potency. Thus, the compositions of this disclosure may be used as preservatives in the preparation of food products, personal care products, or pharmaceutical products to prevent spoilage thereof, for example. Due to the potency of the compositions, the inclusion of minor quantities, e.g., 1% (w/w) or less in a food product, personal care product, or pharmaceutical product suffices. A further attractive feature of the compositions of the present disclosure is that they can be prepared from naturally occurring source materials, notably poultry eggs.

In what follows specific example embodiments are described.

### Compositions

In accordance herewith, in one aspect, the present disclosure provides, in at least one embodiment, a composition comprising phosvitin or peptide fragments thereof, wherein the phosvitin or peptide fragments thereof are substantially free of mineral ions chelated thereto.

The compositions generally comprise phosvitin or peptide fragments thereof, wherein the phosvitin polypeptide or the peptide fragments thereof are substantially free of mineral ions chelated thereto, including, for example, calcium ions (Ca²⁺), iron ions (Fe²⁺), zinc ions (Zn²⁺), and manganese ions (Mn²⁺). Furthermore, the compositions generally can be said to be substantially free of bivalent mineral ions, for example, Ca²⁺, Fe²⁺, Zn²⁺, and Mn²⁺ chelated thereto. In this respect, the phosvitin or fragments thereof included in the compositions of the present disclosure can be distinguished from phosvitin polypeptides occurring in yolk, which are substantially chelated to mineral ions. Notably, the serine residues of phosvitin occurring in yolk are coordinated to chelate mineral ions. In this respect, phosvitin occurring in yolk may comprise, for example, about 295.5 ppm calcium ions (Ca²⁺), about 6.4 ppm iron (Fe²⁺) ions, about 2.6 ppm zinc (Zn²⁺) ions, and 0.044 ppm manganese ions (Mn²⁺), wherein each of these ion species are coordinated to chelate phosvitin.

In one aspect, the present disclosure provides, in one embodiment, compositions comprising phosvitin or peptide fragments thereof, the phosvitin or peptide fragments comprising about 50% or about 50% or less, about 40% or about 40% or less, about 30% or about 30% or less, about 20% or about 20% or less, about 15% or about 15% or less, about 10% or about 10% or less, or about 5% or about 5% or less of the amount of calcium ions (Ca²⁺) chelated to phosvitin occurring in yolk or to peptide fragments thereof.

In one aspect, the present disclosure provides, in one embodiment, compositions comprising phosvitin or peptide fragments thereof, the phosvitin or peptide fragments comprising from about 5% to about 30%, from about 10% to about 25%, or from about 15% to about 20% of the amount of calcium ions (Ca²⁺) chelated to phosvitin occurring in yolk or to peptide fragments thereof.

In one aspect, the present disclosure provides, in one embodiment, compositions comprising phosvitin or peptide fragments thereof, wherein the phosvitin or phosvitin peptide fragments comprise less than about 148 ppm calcium ions (Ca²⁺) chelated thereto, less than about 118 ppm calcium ions (Ca²⁺) chelated thereto, less than about 88.8 ppm calcium ions (Ca²⁺) chelated thereto, less than about 59.2 ppm calcium ions (Ca²⁺) chelated thereto, less than about 44.4 ppm calcium ions (Ca²⁺) chelated thereto, less than about 30.0 ppm calcium ions (Ca²⁺) chelated thereto, or less than less than about 14.8 ppm calcium ions (Ca²⁺) chelated thereto.

In one aspect, the present disclosure provides, in one embodiment, compositions comprising phosvitin or peptide fragments thereof, the phosvitin or peptide fragments comprising about 50% or about 50% or less, about 40% or about 40% or less, about 30% or about 30% or less, about 20% or about 20% or less, about 15% or about 15% or less, about 10% or about 10% or less, or about 5% or about 5% or less of the amount of iron ions (Fe²⁺) chelated to phosvitin occurring in yolk or to peptide fragments thereof.

In one aspect, the present disclosure provides, in one embodiment, compositions comprising phosvitin or peptide fragments thereof, the phosvitin or peptide fragments comprising from about 5% to about 30%, from about 10% to about 25%, or from about 15% to about 20% of the amount of iron ions (Fe²⁺) chelated to phosvitin occurring in yolk or to peptide fragments thereof.

In one aspect, the present disclosure provides, in one embodiment, compositions comprising phosvitin or peptide fragments thereof, wherein the phosvitin or phosvitin peptide fragments comprise less than about 3.2 ppm iron ions (Fe²⁺) chelated thereto, less than about 2.5 ppm iron ions (Fe²⁺) chelated thereto, less than about 1.9 ppm iron ions (Fe²⁺) chelated thereto, less than about 1.4 ppm iron ions (Fe²⁺) chelated thereto, less than about 0.95 ppm iron ions (Fe²⁺) chelated thereto, less than about 0.64 ppm iron ions (Fe²⁺) chelated thereto, or less than less than about 0.32 ppm iron ions (Fe²⁺) chelated thereto.

In one aspect the present disclosure provides, in one embodiment, compositions comprising phosvitin or peptide fragments thereof, the phosvitin or peptide fragments comprising about 50% or about 50% or less, about 40% or about 40% or less, about 30% or about 30% or less, about 20% or about 20% or less, about 15% or about 15% or less, about 10% or about 10% or less, or about 5% or about 5% or less of the amount of calcium ions (Ca²⁺) chelated to phosvitin occurring in yolk or peptide fragments thereof, and, further comprising, about 50% or about 50% or less, about 40% or about 40% or less, about 30% or about 30% or less, about 20% or about 20% or less, about 15% or about 15% or less, about 10% or about 10% or less, or about 5% or about 5% or less of the amount of iron ions (Fe²⁺) chelated to phosvitin occurring in yolk or peptide fragments thereof.

In one aspect, the present disclosure provides, in one embodiment, compositions comprising phosvitin or peptide fragments thereof, the phosvitin or peptide fragments comprising from about 5% to about 30%, from about 10% to about 25%, or from about 15% to about 20% of the amount of calcium ions (Ca²⁺) chelated to phosvitin occurring in yolk or to peptide fragments thereof, and further comprising from about 5% to about 30%, from about 10% to about 25%, or from about 15% to about 20% of the amount of iron ions (Fe²⁺) chelated to phosvitin occurring in yolk or to peptide fragments thereof.

In one aspect the present disclosure provides, in one embodiment, compositions comprising phosvitin or peptide fragments thereof, wherein the phosvitin or phosvitin peptide fragments comprise less than about 148 ppm calcium ions (Ca²⁺) chelated thereto, less than about 118 ppm calcium ions (Ca²⁺) chelated thereto, less than about 88.8 ppm calcium ions (Ca²⁺) chelated thereto, less than about 59.2 ppm calcium ions (Ca²⁺) chelated thereto, less than about 44.4 ppm calcium ions (Ca²⁺) chelated thereto, less than about 30.0 ppm calcium ions (Ca²⁺) chelated thereto, or less than less than about 14.8 ppm calcium ions (Ca²⁺) chelated thereto, and further comprising less than about 3.2 ppm iron ions (Fe²⁺) chelated thereto, less than about 2.5 ppm iron ions (Fe²⁺) chelated thereto, less than about 1.9 ppm iron ions (Fe²⁺) chelated thereto, less than about 1.4 ppm iron ions (Fe²⁺) chelated thereto, less than about 0.95 ppm iron ions (Fe²⁺) chelated thereto, less than about 0.64 ppm iron ions (Fe²⁺) chelated thereto, or less than less than about 0.32 ppm iron ions (Fe²⁺) chelated thereto.

In one aspect, the present disclosure provides, in one embodiment, compositions comprising phosvitin or peptide fragments thereof, wherein the phosvitin or phosvitin peptide fragments comprise less than about 2.3 ppm zinc ions (Zn²⁺) chelated thereto, less than about 2.1 ppm zinc ions (Zn²⁺) chelated thereto, less than about 1.3 ppm zinc ions (Zn²⁺) chelated thereto, less than about 0.65 ppm zinc ions (Zn²⁺) chelated thereto, or less than less than about 0.26 ppm zinc ions (Zn²⁺) chelated thereto.

In one aspect, the present disclosure provides, in one embodiment, compositions comprising phosvitin or peptide fragments thereof, wherein the phosvitin or phosvitin peptide fragments comprise less than about 0.033 ppm manganese ions (Mn²⁺) chelated thereto, less than about 0.022 ppm manganese ions (Mn²⁺) chelated thereto, less than about 0.018 ppm manganese ions (Mn²⁺) chelated thereto, less than about 0.009 ppm manganese ions (Mn²⁺) chelated thereto, or less than less than about 0.004 ppm manganese ions (Mn²⁺) chelated thereto.

In one aspect, the present disclosure provides, in one embodiment, compositions comprising phosvitin or peptide fragments thereof, wherein the phosvitin or peptide fragments comprise less than about 148 ppm calcium ions (Ca²⁺), and less than about 3.2 ppm metallic mineral ions chelated thereto.

In one aspect, the present disclosure provides, in one embodiment, compositions comprising phosvitin or peptide fragments thereof, wherein the phosvitin or peptide fragments comprise less than about 148 ppm calcium ions (Ca²⁺), less than about 3.2 ppm iron ions (Fe²⁺), less than 2.3 ppm zinc ions (Zn²⁺), and less than 0.033 ppm manganese ions (Mn²⁺) chelated thereto.

In one aspect, the present disclosure provides, in one embodiment, compositions comprising phosvitin or peptide fragments thereof, wherein the phosvitin or phosvitin peptide fragments comprise about 53.2 ppm or less calcium ions (Ca²⁺), about 2.6 ppm or less iron ions (Fe²⁺), 2.2 ppm or less of zinc ions (Zn²⁺) and 0.018 ppm or less manganese ions (Mn²⁺) chelated thereto.

In order to prepare phosvitin, poultry eggs, chicken eggs, turkey eggs or goose eggs, for example, may be obtained and used as a source material. Eggs are optionally screened and cleaned if necessary or so desired, to remove extraneous materials such as debris and non-egg materials. Eggs may also optionally be washed, or surface sterilized using, for example, a chemical agent, such as bleach, to reduce contaminating biological agents such as bacteria or fungi that may be present on the exterior eggshell. Eggs then may be broken, and the egg yolks may be separated from the egg white. The egg yolks may be gently mixed using, for example a mechanical stirring device, such as a propeller stirring device operating at medium rates, for example, from about 100 rpm to about 400 rpm, to obtain a more or less homogenous egg yolk preparation. A salt, for example 0.5% (w/w) sodium chloride, may be added to the egg yolks, and the salted egg yolks may be stirred, using for example a mechanical stirring device, for a period of time, for example, for 1 to 3 hours at room temperature. The salted egg yolk mixture then may be subjected to density based separation to recover the insoluble egg yolk protein containing fraction. This may be accomplished, for example, by centrifuging the salted egg yolk mixture at from about 6,000×g to about 7,000×g for about 30 minutes at room temperature or a lower temperature, for example, as low as about 4° C. To the recovered insoluble egg yolk protein containing fraction salt is added, for example, at least about 8 volumes of from about 9% (w/w) to about 11% (w/w) sodium chloride solution, and the pH may be adjusted to approximately neutral. The egg yolk protein fraction then may be stirred for 1 to 3 hours, adjusting the pH to remain approximately neutral as necessary. Thereafter the egg yolk protein preparation may be kept at 4° C. for at least about 6 hours. The egg yolk protein preparation then may again be subjected to a second density based separation to recover the soluble egg yolk protein fraction. This may be accomplished, for example, by centrifuging the egg yolk protein preparation at from about 6,000×g to about 7,000×g for about 30 minutes at room temperature or a lower temperature, for example, as low as about 4° C. The recovered soluble egg yolk protein fraction may then be subjected to treatment with a chelating agent such as ethylene diamine, ethylenediaminetetraacetic acid (EDTA), or ethyleneglycoltetraacetic acid (EGTA), for example, by mixing the soluble egg yolk protein fraction with, for example, from about 0.04 M to about 0.6 M ethylenediaminetetraacetic acid (EDTA), for example, 0.04 M or about 0.04 M EDTA, 0.1 M or about 0.1 M EDTA, 0.2 M or about 0.2 M EDTA, 0.3 M or about 0.3 M EDTA, 0.4 M or about 0.4 M EDTA, 0.5 M or about 0.5 M EDTA, or 0.6 M or about 0.6 M EDTA, for at least about 6 hours, maintaining the pH at approximately neutral pH. The EDTA treated soluble egg yolk fraction then may be dialyzed against distilled water, using for example, 2 to 4 volumes of water and a dialysis membrane having a 5 kDa, or less, molecular weight cut-off. Alternatively, the EDTA treated soluble egg yolk fraction may be subjected to ultrafiltration, using a filter with a 5 kDa, or less, molecular weight cut-off. The dialysate, or the retentate may then be dried, for example, by lyophilization or spray-drying the dialysate or retentate respectively. The obtained dried protein preparation is substantially pure, i.e., the preparation contains at least 80% (w/w), at least 90% (w/w), at least 95% (w/w), at least 96% (w/w), at least 97% (w/w), at least 98% (w/w) or at least 99% (w/w) of phosvitin relative to other egg yolk proteins. Furthermore, the composition comprising phosvitin is substantially free of mineral ions chelated to phosvitin.

Furthermore, where the phosvitin is prepared from chicken eggs, the phosvitin polypeptide comprises SEQ.ID NO: 1. Where phosvitin is prepared from poultry eggs of other species, the phosvitin may be substantially identical to SEQ.ID NO: 1, for example, at least 80%, at least 90%, at least 95%, at least 96%, at least 97%, at least 98%, or at least 99% identical to SEQ.ID NO: 1.

The foregoing techniques and processes allow one to obtain a substantially pure phosvitin preparation or phosvitin peptide fragment preparation, for example, a preparation which is 95% (w/w), 96% (w/w), 97% (w/w), 98% (w/w), or 99% (w/w) pure, wherein the preparation is substantially free of mineral ions chelated to the phosvitin or phosvitin peptide fragment.

Those skilled in this art will understand that the mineral ion content in a phosvitin or phosvitin peptide fragment preparation produced as disclosed herein, can readily be measured by various methodologies known to the art including, for example, inductively coupled plasma mass spectrometric techniques, such as laser ablation inductively coupled plasma mass spectrometric techniques as disclosed by Longerich, H. et al., 1996, J. Analytical Atomic Spectrometry, 11, 899-904.

In another aspect, the phosvitin containing compositions further may be characterized by having a nitrogen (N) content of about 6.3% (w/w) to about 15.2% (w/w), i.e., 6.3% to 15.2% by weight of phosvitin protein.

In another aspect, the phosvitin containing compositions further may be characterized by having a phosphorus (P) content of about 8.5% (w/w) to about 9.5% (w/w), i.e., 8.5% to 9.5% by weight of phosvitin protein.

In another aspect, the phosvitin containing compositions further may be characterized by having a nitrogen to phosvitin ratio (w/w) of from about 2.5 to about 3.7.

Furthermore, in another aspect, dried phosvitin preparation is generally a white or off-white colored fine particulate.

Phosvitin peptide fragments may be prepared using phosvitin extract, for example, a soluble egg yolk protein extract containing phosvitin, or substantially pure phosvitin and by subjecting the phosvitin extract or substantially pure phosvitin preparation to enzymatic digestion or chemical digestion. Chemical digestion may be achieved using for example treatment with hydrochloric acid. In order to enzymatically digest phosvitin, hydrolytic enzymes such as trypsin, chymotrypsin and pepsin, for example, may be used to proteolytically cleave phosvitin polypeptides and obtain phosvitin peptide fragments. Generally, a small quantity of a proteolytic enzyme is mixed with a phosvitin preparation and reacted. The reaction conditions may vary, and the size of the obtained phosvitin proteolytic fragments may be controlled by controlling reaction conditions such as reaction temperature and time. In general, the obtained phosvitin peptide fragments comprising multiple serine residues and at least 10, at least 25, at least 50, at least 75, at least 100, or at least 150 consecutive amino acid residues of SEQ.ID NO: 1 or a polypeptide sequence substantially similar thereto. Thus, for example, phosvitin peptide fragments include, but are not limited to, SEQ.ID NO: 3; SEQ.ID NO: 4; SEQ.ID NO: 5; SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; or SEQ.ID NO: 14, or a peptide fragment having a polypeptide sequence substantially identical thereto, for example, at least 80%, at least 90%, at least 95%, at least 96%, at least 97%, at least 98%, or at least 99% identical to the aforementioned peptide fragments may be prepared.

If desired, upon completion of the reaction the phosvitin peptide fragments may be purified, using protein purification techniques, such as extraction, filtration, and chromatographic techniques generally known to those of skill in the art, and further for example described in: Cutler, P. Protein Purification Protocols, Humana Press, 2004, Second Ed.

Furthermore, as will be known to those of skill in the art, phosvitin and phosvitin peptide fragments may be prepared by recombinantly expressing phosvitin or any desired phosvitin fragment in a host cell, such as an Escherichia coli cell or a yeast cell (e.g., Saccharomyces cerevisiae) and purifying the phosvitin polypeptide or peptide fragments from the host cell culture. Phosvitin or peptide fragments thereof obtained from a host cell culture may be subjected to treatment with a chelating agent to obtain a composition comprising phosvitin or phosvitin peptide substantially free of mineral ions chelated to the phosvitin peptide fragments. In what follows next aspects of techniques for recombinant production of phosvitin and phosvitin peptide fragments are discussed further.

Thus, as is known to those of skill in the art, expression of nucleic acids in a host cell, to thereby biosynthetically produce a protein, can be achieved by providing one or more nucleic acids capable of controlling expression in a host cell, and operably linking the one or more nucleic acids capable of controlling expression in a host cell to the nucleic acid one wishes to express. Such operable linking of a nucleic acid controlling expression generally involves linking in the 5′ to 3′ direction of expression the nucleic acid capable of controlling expression in a host cell to the nucleic acid one wishes to express. Thus, within the context of the instant disclosure, a nucleic acid encoding a phosvitin polypeptide, including, for example SEQ.ID NO: 2 can be linked to a nucleic acid controlling expression in a host cell. Suitable nucleic acid sequences capable of controlling expression in host cells that may be used herein include any transcriptional promoter capable of controlling expression of polypeptides in host cells. Generally, promoters obtained from bacterial cells are used when a bacterial host cell is selected, while a yeast promoter will be used when a yeast host cell is selected. The obtained nucleic acid comprising a promoter and the nucleic acid expressing phosvitin or a fragment thereof is generally a chimeric nucleic acid. Further nucleic acid elements capable elements of controlling expression in a host cell include transcriptional terminators, enhancers and the like, all of which may be included in the chimeric nucleic acid sequences of the present disclosure.

The chimeric nucleic acid sequences including a nucleic acid sequence expressing a phosvitin polypeptide, such as SEQ:ID NO: 1, or a fragment thereof, such as, SEQ.ID NO: 3, SEQ.ID NO: 4, SEQ.ID NO: 5, SEQ.ID NO: 6, SEQ.ID NO: 7, SEQ.ID NO: 8, SEQ.ID NO: 9, SEQ.ID NO: 11, SEQ.ID NO: 12, SEQ.ID NO: 13 or SEQ.ID NO: 14, for example, can be integrated into a recombinant expression vector which ensures good expression in the host cell, wherein the recombinant expression vector is suitable for expression in a host cell. The term “suitable for expression in a host cell” means that the recombinant expression vector comprises the chimeric nucleic acid linked to genetic elements required to achieve expression in a cell. As noted, such genetic elements can include transcriptional promoters, terminators and enhancers, and the like. Further genetic elements that may be included in the expression vector are one or more nucleic acid sequences encoding marker genes, and one or more origins of replication. In some embodiments, the expression vector can freely replicate in the host cell. In other embodiments, the chimeric nucleic acid can be integrated into the host cell's genomic DNA. In some embodiments, the expression vector further can comprise genetic elements required for the integration of the vector or a portion thereof in the host cell's genome.

Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin, chloramphenicol, methotrexate, or ampicillin. In other instances, a marker gene may be a gene which allows a cell to produce an essential nutrients, for example amino acids.

Turning now to the host cell, it is noted, initially, that any host cell which upon cultivation expresses the nucleic acid sequence encoding a phosvitin polypeptide or a fragment thereof can be selected and used. Suitable host cells in this respect include, for example, microbial cells, such as bacterial cells, yeast cells, for example, and algal cells or animal cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

One example host cell that conveniently may be used is Escherichia coll. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (Ipp) promoter, and A phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well-known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces, Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZa, pHIL-D2, pHIL-S1, pPIC3.5K, pPIC9K, pPICZ, pPICZa, pPIC3K, pHWO10, pPUZZLE and 2 μm plasmids. Such vectors are known to the art and are, for example, described in Cregg et al., Mol Biotechnol. (2000) 16(1): 23-52. Suitable promoter sequences for use in yeast host cells are also known and described, for example, in Mattanovich et al., Methods Mol. Biol., 2012, 824:329-58, and in Romanos et al., 1992, Yeast 8: 423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URAS, URA6), histidine (HIS3), and the like. Methods for introducing vectors into yeast host cells can, for example, be found in S. Kawai et al., 2010, Bioeng. Bugs 1(6): 395-403.

A further example of host cells that may be used in accordance herewith are animal host cells. These include, for example, mammalian cells, such as Chinese Hamster Ovary cells (CHO) cells, or lymphoid cells (e.g., YO, NSO, or Sp20 cells), which are able to grow and survive when placed in either monolayer culture or suspension culture in medium containing appropriate nutrients and/or growth factors. Examples of expression vectors suitable for expression in animal cells include, but are not limited to, BPV-1, pHyg, pRSV, pIRES (Clontech), and pSG5 vectors (Stratagene). Selectable markers can used in to confer resistance to the cells harboring the vector to allow their selection in appropriate selection medium. A number of selection systems can be used, including but not limited to, the Herpes Simplex Virus thymidine kinase (HSV TK), (Wigler et al., 1977, Cell, 11:223), hypoxanthine-guanine phosphoribosyltransferase (HGPRT), (Szybalska and Szybalski, 1992, Proc. Natl. Acad. Sci. USA, 48:202), and adenine phosphoribosyltransferase (Lowy et al., 1980, Cell, 22:817) genes. Further vectors, media and growth conditions for animal cells can be found in Sambrook et al., Molecular Cloning, a Laboratory Manual, Cold Spring Harbor Laboratory Press, 2012, Fourth Ed.

Further, guidance with respect to the preparation of expression vectors and introduction thereof into host cells, may be found in, for example: Sambrook et al., Molecular Cloning, a Laboratory Manual, Cold Spring Harbor Laboratory Press, 2012, Fourth Ed.

Host cells can be grown to multiply and to express a chimeric nucleic acid. Expression of the chimeric nucleic acid results in the biosynthetic production in the host cell of a phosvitin polypeptide or fragments thereof. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Typical carbon sources are e.g., mono- or disaccharides. Typical nitrogen sources are, e.g., ammonia, urea, amino acids, yeast extract, corn steep liquor and fully or partially hydrolyzed proteins. Typical trace metals are e.g., Fe, Zn, Mn, Cu, Mo and H₃BO₃. Typical water soluble vitamins are e.g., biotin, pantothenate, niacin, thiamine, p-aminobenzoic acid, choline, pyridoxine, folic acid, riboflavin and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation, and CD-CHO medium, or Ham's F10 medium for growing CHO cells. Further media and growth conditions can be found in Sambrook et al., Molecular Cloning, a Laboratory Manual, Cold Spring Harbor Laboratory Press, 2012, Fourth Ed.

Upon production by the host cells of a phosvitin polypeptide or a fragment thereof, the phosvitin polypeptide or fragment thereof may be recovered from the host cells, and separated from other constituents, such as cellular debris, or media constituents, for example. Separation techniques will be known to those of skill in the art and include a variety of different protein purification techniques including, e.g., ion-exchange chromatography, size exclusion chromatography, affinity chromatography, hydrophobic interaction chromatography, reverse phase chromatography, gel filtration, etc. Further general guidance with respect to protein purification may, for example, be found in: Cutler, P. Protein Purification Protocols, Humana Press, 2004, Second Ed.

The recovered recombinant phosvitin protein protein or peptide fragments thereof may then be subjected to treatment with a chelating agent, for example, by mixing the protein with from about 0.04 M to about 0.6 M ethylenediaminetetraacetic acid (EDTA), for at least about 6 hours, maintaining the pH at approximately neutral pH. The EDTA treated recombinant phosvitin protein of peptide fragments hen may be dialyzed against distilled water, using for example, 2 to 4 volumes of water and a dialysis membrane having a 5 kDa, or less, molecular weight cut-off. Alternatively, the EDTA treated recombinant phosvitin or peptide fragments thereof may be subjected to ultrafiltration, using a filter with a 5 kDa, or less, molecular weight cut-off. The dialysate, or the retentate may then be dried, for example, by lyophilization or spray-drying the dialysate or retentate respectively.

Thus, the foregoing techniques allow one to obtain substantially pure preparations of phosvitin polypeptides or fragments thereof. The recovered phosvitin polypeptide or peptide fragments thereof may be obtained in a more or less pure form, for example, a preparation of phosvitin polypeptides or fragments thereof of at least about 60% (w/v), about 70% (w/v), about 80% (w/v), about 90% (w/v), about 95% (w/v), or about 99% (w/v) purity may be obtained.

In one embodiment, the phosvitin or peptide fragments thereof can constitute at least about 95% (w/w), at least about 96% (w/w), at least about 97% (w/w), at least about 98% (w/w), or at least about 99% (w/w) of the preparation.

In one embodiment, the phosvitin or peptide fragments thereof can constitute at least about 95% (w/w), at least about 96% (w/w), at least about 97% (w/w), at least about 98% (w/w), or at least 99% about (w/w) of the preparation, and the preparation comprises no more than about 148 ppm calcium ions (Ca²⁺), and no more than about 3.2 ppm metallic mineral ions chelated thereto.

In one embodiment, the phosvitin or peptide fragments thereof can constitute at least about 95% (w/w), at least about 96% (w/w), at least about 97% (w/w), at least about 98% (w/w), or at least about 99% (w/w) of the preparation, and the preparation comprises no more than 148 ppm calcium ions (Ca²⁺), less than about 3.2 ppm iron ions (Fe²⁺), less than 2.3 ppm zinc ions (Zn²⁺), and less than 0.033 ppm manganese ions (Mn²⁺) chelated thereto.

In one embodiment, the pH of the preparation can be adjusted to a selected pH. Thus, for example, the pH can be set to a selected pH in the range of from about 5.0 to about 8.0, for example using a 50% (v/v) NaOH solution, or HCl to adjust the pH.

In one embodiment, the form preparation can be buffered to maintain the selected pH. Thus, for example, a phosphate buffered saline (PBS) pH 7.4 buffer may be used (0.137 M NaCl; 0.027 M KCl; 0.01 M Na₂HPO₄—; and 0.0018 M KH₂PO₄) to maintain a selected pH.

### Preservative Formulations

The obtained preparations comprising phosvitin or phosvitin peptide fragments may be mixed with a diluent, carrier, or auxiliary agent, to obtain a formulation, comprising phosvitin or phosvitin peptide fragments and a diluent, carrier, or auxiliary agent. The obtained formulations may be used as a preservative agent, and thus can be said to be preservative formulations. Thus, in accordance with one aspect of the disclosure, provided herein are preservative formulations comprising phosvitin or phosvitin peptide fragments, wherein the phosvitin or phosvitin fragments are substantially free of mineral ions chelated thereto.

In some embodiments, the diluent can be water, or a buffer, such as, for example, a phosphate buffered saline (PBS) buffer, and liquid phosvitin formulations comprising a range of phosvitin dilutions may be prepared, for example, liquid formulations comprising phosvitin or phosvitin peptide fragments in a concentration of about 250 mg/ml or less, 100 mg/ml or less, 10 mg/ml or less, about 5 mg/ml or less, about 1 mg/ml or less, or 0.1 mg/ml or less.

In one embodiment, the carrier can be a phospholipid, including a glycerophospholipid (e.g., phosphatidyl choline (PC), a phosphatidyl ethanolamine (PE), phosphatidylserine (PS), phosphatidic acid (PA), phosphatidyl inositol (PI), phosphatidyl glycerol (PG), or cardiolipin), or a sphingomyelin.

A wide range of auxiliary agents may be included in the preservative formulations. Thus, for example, auxiliary agents that may be included in the preservative formulations of the present disclosure include, for example, antimicrobial peptides, chelating agents, essential oils, photochemically active agents, cannabinoids, terpenes, bitter acids, polyphenols and vitamins, or mixtures thereof.

Antimicrobial peptides that may be included in the preservative formulations are, for example, bacteriocins (e.g., nisin, lactosin S, epidermin, gallidermin, lacticin 481, mersacidin, cinnamycin, ancovenin, duramycin, actagardin, pediocin PA-1, pediocin AcH, sakacin A, sakacin P, leucocin A-UAL 187, leucocin F10, mesentericin Y105, enterocin A, divercin V41, lactococcin MMFII, lactococcin G, lactoccin M, lactacin F, lacticin 3147A, lacticin 3147B, plantaricin A, plantaricin C, plantaricin EF, plantaricin JK, acidocin A, acidocin B., acidocin J1132, carnobacteriocin A, divergicin A, enterocin P, enterocin B, helvitcicin J, helviticin V-1829, thermophilin 13, bavaracin A, curvacin A, divercin V41, mesentericin Y105, mundticin, piscicocin Via, piscicocin V1b, or piscicolin 126) cathelicidins (e.g., LL 37), defensins (e.g., α-defensins, δ-defensins or θ-defenesins), antimicrobial enzymes (e.g., lysozyme, lactoperoxidase, or phospholipase A2), bactofencin (e.g., bactofencin A), laterosporulin, or mixtures thereof.

Chelating agents that may be included in the preservative formulations are, for example, lactoferrin, ovotransferrin, deferoxamine, desferthiocin, deferiprone, clioquinol, O-trensox, deferasirox, tachpyr, dexrazoxane, triapine, pyridoxal, di-pyridylketone, flavan-3-ol, curcumin, apocynin, kolavirin, floranol, baicalein, baicalin, ligustrazine, quercetin, epigallocatechin gallate, theaflavin, or mixtures thereof.

Essential oils that may be included in the preservative formulations are, for example, anise oil, atlas cedar oil, balsam fir oil, basil oil, bergamot oil, black pepper oil, camphor oil, cardamom oil, carrot seed oil, cedar wood oil, chamomile oil, cinnamon bark oil, cinnamon cassia oil, citronella oil, clary sage oil, clove oil cypress oil, eucalyptus oil, eucalyptus radiata oil, frankincense oil, geranium oil, ginger oil, grapefruit oil, hop oil, hyssop oil, juniper berry oil, lavender oil, lemon oil, lemon eucalyptus oil, lemon grass oil, lime oil, marjoram oil, myrrh oil, nutmeg oil, orange oil, oregano oil, patchouli oil, pennyroyal oil, peppermint oil, pine oil, rosemary oil, sage oil, spearmint oil, spike lavender oil, tangerine oil, tea tree oil, thyme oil, vetiver oil, wintergreen oil, ylang ylang oil, or mixtures thereof.

Photochemically active agents that may be included in the preservative formulations are, for example, flavonoids, flavanols, flavanones, flavones, isoflavanones, anthocyanidins, gallic acid, catechins, quercetin glucoside, flavon-3-ols, or mixtures thereof.

Vitamins that may be included in the preservative formulations are, for example, vitamin A, vitamin B1, vitamin B2, vitamin B3, vitamin B5, vitamin B6, vitamin B7, vitamin B9, vitamin B12, vitamin C, vitamin D, vitamin E, vitamin K, or mixtures thereof.

Terpenes that may be included in the preservative formulations are, for example, limonene, limonene-oxide, myrcene, linalool, carophyllene, α-pinene, β-pinene, α-bisabolol, terpinene, eucalyptol, trans-nerolido, humulene, δ-3-carene, camphene, borneol, terpineol, valencene, geraniol, α-humulene, β-carophyllene, β-farnesene, δ-cadinene, α-selinene, β-selinene, γ-muurolene, myrcenol, carophyllene oxide, farnesol, or mixtures thereof.

Cannabinoids that may be included in the preservative formulations are, for example, cannabigerolic acid (CBGA), δ-9-tetrahydrocannabinolic acid (THCA), cannabidiolic acids (CBDA), cannabichromenic acid (CBCA), cannabigerovarinic acid (CBGVA), tetrahydrocanabivarinic acid (THCVA), cannabidivarinic acid (CBDVA), cannabichromevarinic acid (CBCVA), cannabigerol acid (CBG), δ-9-tetrahydrocannabinol (THC), cannabidiol (CBD), cannabichromene (CBC), cannabigerovarine (CBGV), tetrahydrocanabivarin (THCV), cannabidivarin (CBDV), cannabichromevarin acid (CBCV), cannabinol, or mixtures thereof.

Bitter acids that may be included in the preservative formulations include, for example, α-bitter acids (e.g., cohumulone, humulone, prehumulone, posthumulone, adhumulone, or adprehumulone), δ-bitter acids (e.g., colupulone, lupulone, perlupulone, postlupulone, or adlupulone), or mixtures thereof.

Polyphenols that may be included in the preservative formulations include, for example, (+)-catechin, (−)epicatechin, (+)-gallocatechin, procyanidin B3, prodelphinidin B3, kaempferol, quercitin, myericetin, morin, rutin, quercitrin, naringenin, xanthohumol, desmethylxanthohumol, isoxanthohumol, 8-prenylnaringenin, 6-prenylnaringenin, benzoic acid derivatives (e.g., p-hydroxybenzoic acid, protocatechuic acid, gallic acid, or vanillic acid), cinnamic acid derivatives (e.g., p-coumaric acid, caffeic acid, ferulic acid, or sinapic acid), or mixtures thereof.

In general, in order to prepare the preservative formulations, a preparation containing demineralized phosvitin, or a fragment thereof can be mixed with a diluent, carrier, or auxiliary agent by contacting the ingredients and mixing them. For example, the ingredients may together be introduced in a mixing vessel, a beaker, for example, and mixing the ingredients at room temperature using a stirring device or homogenizing device, such as a handheld electrically powered homogenizer or laboratory blender, until a substantially homogenous formulation is obtained.

In one embodiment, the phosvitin or peptide fragments thereof together with the diluent, carrier, or auxiliary agent, can constitute from at least about 10% (w/w) to about 95% (w/w), for example, about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), or about 95% (w/w) of the formulation, and the formulation comprises no more than 148 ppm calcium (Ca²⁺) ions, and no more than 3.2 ppm metallic mineral ions chelated thereto, the balance of the formulation comprising, substantially consisting of, or consisting of one or more of a diluent, carrier, or an auxiliary agent.

In one embodiment, the phosvitin or peptide fragments thereof together with the diluent or carrier, can constitute from at least about 10% (w/w) to at least about 95% (w/w), of the formulation, for example, about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), or about 95% (w/w) and the formulation comprises no more than 148 ppm calcium ions (Ca²⁺), less than about 3.2 ppm iron ions (Fe²⁺), less than 2.3 ppm zinc ions (Zn²⁺), and less than 0.033 ppm manganese ions (Mn²⁺) chelated thereto, the balance of the formulation comprising, substantially consisting of, or consisting of one or more of a diluent, carrier, or an auxiliary agent.

### Products

The above described compositions can be used to formulate products that require a preservative, including but not limited to, food products, personal care products, or pharmaceutical products.

In another aspect, the present disclosure relates to products, and methods of making products containing phosvitin. Accordingly, the present disclosure further provides, in at least one embodiment, a method comprising:
(i) providing a composition comprising phosvitin or peptide fragments thereof, wherein the phosvitin or peptide fragments thereof are substantially free of mineral ions chelated thereto; and
(ii) formulating an effective preservative amount of the composition with ingredients suitable for formulating the product, to thereby form the product.

In an aspect, according to some embodiments of the present disclosure, the phosvitin or phosvitin peptide fragment containing compositions described herein may be used as ingredients in food products. Food products in this respect are any products for human consumption providing nutritional value, and include any foods, beverages, dietary supplements, and nutraceuticals. Food products include, without limitation, any grain based product (e.g., bread, breakfast cereal, pasta, and so on) dairy product (e.g., yoghurt, ice cream, cheese, infant formula, and so on), egg or egg ingredient containing product (e.g., mayonnaise, creamy salad dressing, baked goods, and so on), and fruit or vegetable based product, or fruit or vegetable ingredient containing product (e.g., tomato paste, fruit juice, plant-protein based meat alternative, and so on). In order to prepare food products, the compositions described herein may be contacted with or blended with or mixed together with formulary ingredients suitable for preparing a food product, including, for example, animal plant oils or fats, animal or plant proteins, dietary fiber, carbohydrates, sugars, water, and auxiliary agents, such as colorants, flavorants, viscosity modifying agents, pH modifying agents, and the like.

In an aspect, according to some embodiments of the present disclosure, the phosvitin or phosvitin peptide fragment containing compositions described herein may be used as ingredients in personal care products. Personal care products in this respect are any products suitable for cleaning, cleansing, maintaining, protecting, repairing, remediating or modifying, the appearance or beautifying the exterior surface of the human body, including, skin, hair, nails, teeth and lips, by topical application of the product to the surface area of the human body. Personal care products include, without limitation, skin care products (e.g., a skin cream, facial cream, anti-wrinkle cream, facial mask, and so on), hair care products (e.g., a shampoo, a conditioner, a hair dye, a pomade, and so on), a bath and body formulation (e.g., a body wash, a bar soap, a shower gel, and so on), a sun care formulation (e.g., a sunscreen, an after sun lotion, a tanning lotion, and so on), a make-up (e.g., a mascara, a lipstick, a blush, and so on). In order to prepare personal care products, the compositions described herein may be contacted with or blended with or mixed together with formulary ingredients suitable for preparing a personal care product, including, for example, oils, lipids, waxes, moisturizers, excipients, including surface active agents, such as anionic or cationic surfactants, diluents, and auxiliary agents, such as colorants, viscosity modifying agents, skin active agents, pH modifying agents, and the like.

In an aspect, according to some embodiments of the present disclosure, the phosvitin or phosvitin peptide fragment containing compositions described herein may be used as ingredients in pharmaceutical products. Pharmaceutical products in this respect are any medicinal or therapeutic products to treat or prevent the development of a medical condition, disease or disorder. Pharmaceutical products include, without limitation, tablets, syrups, capsules, lozenges, sprays, creams, suppositories, gels, suspensions, and solutions. In order to formulate pharmaceutical products, the compositions described herein may be contacted with or blended with or mixed together with formulary ingredients suitable for preparing a pharmaceutical product, including, at least a pharmaceutically active agent, and further including, for example, a diluent, excipient, carrier, or auxiliary agent such as binders, surface active agents, colorants, flavorants, viscosity modifying agents, pH modifying agents, and the like.

In some embodiments, a food product, personal care product, or pharmaceutical product may be pre-formed, and the composition comprising phosvitin or peptide fragments thereof may be separately provided and incorporated in an effective preservative amount into the pre-formed food product, personal care product, or pharmaceutical product.

The manner of incorporation of the phosvitin or phosvitin peptide fragments containing composition in the food product, personal care product, or pharmaceutical product, or other products, may vary. In general, phosvitin or phosvitin peptide fragments containing composition may be more or less homogenously distributed within and throughout the formed product, such as the food product, personal care product, or pharmaceutical product.

In some embodiments, the phosvitin or phosvitin peptide fragments containing composition may be incorporated during preparation of the product food product, such as the personal care product, or pharmaceutical product. In such embodiments, the phosvitin or phosvitin peptide fragments containing composition may be added separately or alternatively, the composition may be incorporated together with one or more other formulary compounds to form the product.

The final concentration of the phosvitin or phosvitin peptide fragments containing composition in the formed product, such as the food product, personal care product, or pharmaceutical product, may vary. In some embodiments, the phosvitin or phosvitin peptide fragments containing composition may comprise no more than about 5% (w/w), 4% (w/w), 3% (w/w), 2% (w/w), 1% (w/w), 0.5% (w/w), 0.2% (w/w), or 0.1% (w/w) of the product, such as the food product, personal care product, or pharmaceutical product. The concentration of the phosvitin or phosvitin peptide fragments containing composition may be optimized or adjusted by preparing a plurality of products, wherein each product comprises a different concentration of the phosvitin or phosvitin peptide fragments containing composition, then evaluating each of the products with reference to one or more effects, then selecting one or more for the products to provide a selected desirable effect.

It is noted that the composition comprising phosvitin or phosvitin peptide fragments can be used as a preservative agent in products, including, for example, a food product, personal care product, or pharmaceutical product. In this respect, the composition comprising substantially pure phosvitin or phosvitin peptide fragments when formulated with other ingredients suitable to formulate a product, such as a food product, personal care product, or pharmaceutical product, for example, may prevent or restrict oxidation reactions and/or microbial proliferation in the product, and thereby decrease product spoilage and increase product shelf life. Generally, the product is prepared to include an effective preservative amount of the composition. Effective amounts can include amounts sufficient to prevent or delay spoilage of the product or amounts sufficient to extend the shelf life of the product. Effective amounts can vary depending on the ingredients used to formulate the product, for example, the food product, personal care product, or pharmaceutical product or the conditions to which the product are exposed, and can be determined by those of skill in the art. Thus, for example, a series of food products, personal care products, or pharmaceutical products comprising various amounts of a composition of the present disclosure may be prepared and evaluated for spoilage over time. Effective preservative amounts can include, for example, amounts that can extend the shelf life of a product by at least 3 months, at least 6 months, or at least 9 months, when compared to a product not including the composition. In this manner the compositions of the present disclosure can be said to be preservatives.

Without wishing to be bound by theory, it is noted that the compositions of the present disclosure may exhibit anti-oxidant properties and/or antimicrobial properties, and by virtue of such properties act as a preservative.

In some embodiments, an effective preservative amount may be an amount wherein a product, a food product, personal care product, or pharmaceutical product, for example, contains 2% (w/w) or less, 1% (w/w) or less, 0.5% (w/w) or less, 0.2% (w/w) or less, 0.2% (w/w) or less, or 0.1% (w/w) or less of the composition containing phosvitin or phosvitin peptide fragments.

Thus, it will be clear from the foregoing that the present disclosure, in another aspect relates to products, including without limitation, food products, personal care products and pharmaceutical products containing phosvitin. Accordingly, the present disclosure further provides, in at least one embodiment, a food product, personal care product, or pharmaceutical product comprising:
(i) an effective preservative amount of a composition comprising phosvitin or peptide fragments thereof, wherein the phosvitin or peptide fragments thereof are substantially free of mineral ions chelated thereto; and
(ii) additional ingredients suitable for formulating a product, to thereby form the product.

In at least one embodiment, the phosvitin or peptide fragments thereof can constitute at least about 95% (w/w), at least about 96% (w/w), at least about 97% (w/w), at least about 98% (w/w), or at least about 99% (w/w) of the composition, and the composition comprises no more than about 148 ppm calcium ions (Ca²⁺), and no more than about 3.2 ppm metallic mineral ions chelated thereto, and wherein the product, such as the food product, personal care product, or pharmaceutical product, for example, contains no more than about 1% (w/w) of the composition.

In at least one embodiment, the phosvitin or peptide fragments thereof can constitute at least about 95% (w/w), at least about 96% (w/w), at least about 97% (w/w), at least about 98% (w/w), or at least about 99% (w/w) of the composition, and the composition comprises less than about 148 ppm calcium ions (Ca²⁺), less than about 3.2 ppm iron ions (Fe²⁺), less than 2.3 ppm zinc ions (Zn²⁺), and less than 0.033 ppm manganese ions (Mn²⁺) chelated thereto, and wherein the product, such as the food product, personal care product, or pharmaceutical product, for example, contains no more than about 1% (w/w) of the composition.

In yet another aspect, the present disclosure relates to uses of phosvitin. Thus, in an aspect, the present disclosure provides, in at least one embodiment, a use of a composition comprising phosvitin or peptide fragments thereof, wherein the phosvitin or peptide fragments thereof are substantially free of mineral ions bound thereto in the manufacture of a product, including without limitation, a food product, personal care product, or pharmaceutical product.

In at least one embodiment, the phosvitin or peptide fragments thereof can constitute at least about 95% (w/w), at least about 96% (w/w), at least about 97% (w/w), at least about 98% (w/w), or at least about 99% (w/w) of the composition, and the composition comprises no more than about 148 ppm calcium ions (Ca²⁺), and no more than about 3.2 ppm metallic mineral ions chelated thereto, and wherein the product, such as the food product, personal care product, or pharmaceutical product, for example, contains no more than about 1% (w/w) of the composition.

In at least one embodiment, the phosvitin or peptide fragments thereof can constitute at least about 95% (w/w), at least about 96% (w/w), at least about 97% (w/w), at least about 98% (w/w), or at least about 99% (w/w) of the composition, and the composition comprises less than about 148 ppm calcium ions (Ca²⁺), less than about 3.2 ppm iron ions (Fe²⁺), less than 2.3 ppm zinc ions (Zn²⁺), and less than 0.033 ppm manganese ions (Mn²⁺) chelated thereto, and wherein the product, such as the food product, personal care product, or pharmaceutical product, for example, contains no more than about 1% (w/w) of the composition.

Thus, it will now be clear that the present disclosure provides compositions comprising phosvitin or peptide fragments thereof, wherein the phosvitin or peptide fragments thereof are substantially free of mineral ions chelated thereto, and methods of preparing and using such compositions.

### SUMMARY OF SEQUENCES

SEQ.ID NO: 1 sets forth a chicken phosvitin polypeptide sequence.

SEQ.ID NO: 2 sets forth a polynucleotide sequence encoding a chicken phosvitin polypeptide.

SEQ.ID NO: 3 sets forth a chicken phosvitin polypeptide fragment sequence.

SEQ.ID NO: 4 sets forth another chicken phosvitin polypeptide fragment sequence.

SEQ.ID NO: 5 sets forth another chicken phosvitin polypeptide fragment sequence.

SEQ.ID NO: 6 sets forth another chicken phosvitin polypeptide fragment sequence.

SEQ.ID NO: 7 sets forth another chicken phosvitin polypeptide fragment sequence.

SEQ.ID NO: 8 sets forth another chicken phosvitin polypeptide fragment sequence.

SEQ.ID NO: 9 sets forth another chicken phosvitin polypeptide fragment sequence.

SEQ.ID NO: 10 sets forth another chicken phosvitin polypeptide fragment sequence.

SEQ.ID NO: 11 sets forth another chicken phosvitin polypeptide fragment sequence.

SEQ.ID NO: 12 sets forth another chicken phosvitin polypeptide fragment sequence.

SEQ.ID NO: 13 sets forth another chicken phosvitin polypeptide fragment sequence.

SEQ.ID NO: 14 sets forth another chicken phosvitin polypeptide fragment sequence.

Hereinafter are provided examples of specific embodiments of the compositions of the present disclosure and methods of the present disclosure. The examples are provided for illustrative purposes only, and are not intended to limit the scope in any way.

### EXAMPLES

### Example 1

### Method for Making a Phosvitin Preparation Substantially Free of Mineral Ions Chelated Thereto

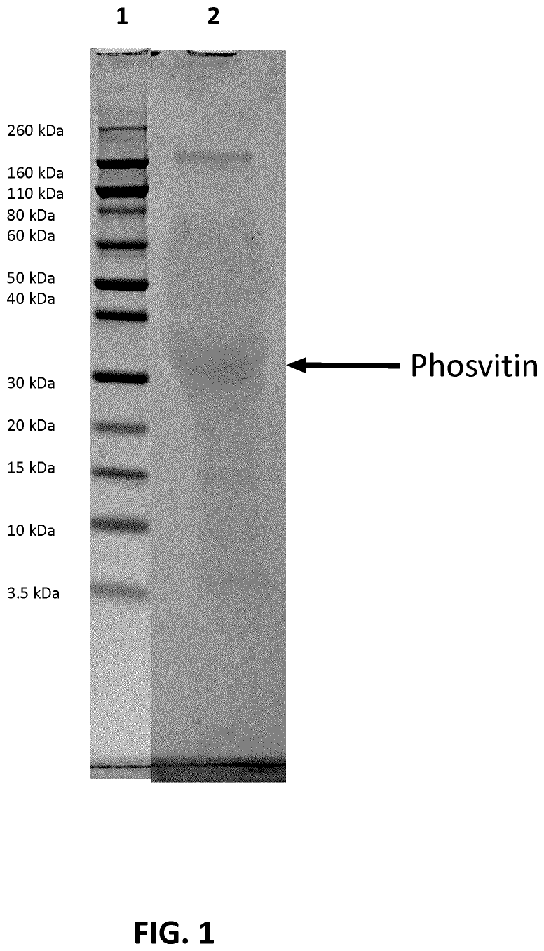

An egg yolk preparation was obtained by breaking 24 cleaned chicken eggs and separating egg whites from egg yolks and gently stirring the egg yolks in a Pyrex glass beaker at room temperature using a propeller stirring device operated at 300 rpm until a homogenous egg yolk preparation was obtained. A total of 0.5% (w/w) sodium chloride was added to the preparation and gentle stirring at room temperature was continued for another 2 hours. The egg yolk preparation was then centrifuged at 6,500×g for 30 minutes at room temperature. The pellet was recovered from the centrifuge vessel and 8 volumes of a 10.1% (w/w) sodium chloride solution was added, after which the pH was adjusted to about 7.0. The mixture was then gently stirred in a Pyrex glass beaker at room temperature using a propeller stirring device operated at 300 rpm for 2 hours while the pH was maintained at 7.0 by periodic pH measurement and adjustment. Thereafter the mixture was stored at 4° C. for a period of 6 hours and then centrifuged at 6,500×g for 30 minutes at room temperature. Approximately half of the supernatant comprising soluble egg yolk proteins was recovered from the centrifuge vessel and treated with 0.5 M ethylenediaminetetraacetic acid (EDTA) for 6 hours in a Pyrex glass beaker at room temperature using a propeller stirring device operated at 300 rpm maintaining the pH at pH 7.0. Both the non-EDTA treated and the EDTA-treated soluble egg yolk protein preparations were then dialyzed against 3 volumes of distilled water using a 3.5 kDa cut-off filter. The dialyzed preparations were then spray dried. The EDTA-treated spray dried protein preparation was subsequently analyzed using SDS gel-electrophoresis as shown in FIG. 1. As can be seen in FIG. 1, the EDTA-treated spray dried egg yolk protein preparation contained substantially pure (at least 80%) phosvitin.

Both the EDTA treated and non-EDTA treated spray dried phosvitin containing protein preparations were analyzed with respect to the mineral ion content using laser ablation inductively coupled plasma mass spectrometry.

Table 1 shows the mineral concentrations of both EDTA-treated and non-EDTA-treated phosvitin containing protein preparations.

TABLE 1
Mineral Content of Phosvitin Containing Protein Preparations

| Mineral | Concentration (Non-EDTA) | | Concentration (EDTA) | |
|---|---|---|---|---|
| Calcium (Ca²⁺) | 295.90 | ppm | 53.24 | ppm |
| Iron (Fe²⁺) | 6.35 | ppm | 2.56 | ppm |
| Zinc (Zn²⁺) | 2.611 | ppm | 2.18 | ppm |
| Manganese (Mn²⁺) | 0.044 | ppm | 0.018 | ppm |

### Example 2

### Preparation of a Mayonnaise Using a Phosvitin Containing Protein Preparation Substantially Free of Mineral Ions

Two mayonnaises were prepared using the phosvitin containing protein preparations described in Example 1, using the following ingredients: 10 g of 10% salted egg yolk, 7.18% water, 0.5% salt, 7.0% white vinegar (5% acidity), 0.2% phosvitin (mayonnaise #1 non-EDTA treated; mayonnaise #2, EDTA-treated), and 75.0% plant oil. The egg yolk was combined with water and salt in a 250 mL Pyrex glass beaker. The solution was stirred propeller stirring device operated at 300 rpm and left standing for 10 min with occasional stirring to promote mixing. The egg mixture was then transferred to a food processor with a blade (Hamilton Beach Prep Star 350W max, used at setting #2). The egg mixture was blended for 1 min, and then 60% of the plant oil was added through a plastic funnel. The remaining plant oil (19%) was mixed with the 7.0% vinegar to acidify the emulsion and incorporated into the blend using the food processor. Once the oil/vinegar mixture was incorporated it was blended using the food processor for an additional one minute to obtain the mayonnaise. The shelf-life stability of mayonnaise #1 and mayonnaise #2 was evaluated by storing both mayonnaise formulations in a closed jar at room temperature for 9 months. Thereafter, the mayonnaise formulations were inspected. Following 9 months of storage mayonnaise #1 was found to have physically separated, i.e., the emulsion was broken, and mayonnaise #1 was malodorous indicative of oxidative instability. By contrast, mayonnaise #2 was found to be a stable emulsion and to not be malodorous.

1. A method for formulating a product comprising:
(i) providing a composition comprising phosvitin or peptide fragments thereof, wherein the phosvitin or peptide fragments thereof are substantially free of mineral ions chelated thereto; and
(ii) formulating an effective preservative amount of the composition with additional ingredients suitable for formulating the product, to thereby form the product.

2. A method according to claim 1, wherein the product is a food product, personal care product or pharmaceutical product.

3. A method according to claim 2, wherein the phosvitin or peptide fragments thereof are substantially free of bivalent mineral ions chelated thereto.

4. A method according to claim 2, wherein the phosvitin or peptide fragments thereof are substantially free of bivalent mineral ions chelated thereto, the mineral ions selected from the group consisting of calcium ions (Ca²⁺), iron ions (Fe²⁺), zinc ions (Zn²⁺), and manganese ions (Mn²⁺).

5-9. (canceled)

10. A method according to claim 2, wherein the phosvitin or peptide fragments thereof comprise less than about 148 ppm calcium ions (Ca²⁺), less than about 3.2 ppm iron ions (Fe²⁺), less than 2.3 ppm zinc ions (Zn²⁺), and less than about 0.033 ppm manganese ions (Mn²⁺) chelated thereto.

11. A method according to claim 2, wherein the phosvitin or peptide fragments thereof, comprise about 53.2 ppm or less calcium ions (Ca²⁺), about 2.6 ppm or less iron ions (Fe²⁺), about 2.2 ppm or less zinc ions (Zn²⁺), and about 0.018 ppm or less manganese ions (Mn²⁺) chelated thereto.

12. A method according to claim 2, wherein the phosvitin or peptide fragments thereof constitute at least about 95% (w/w), at least about 96% (w/w), at least about 97% (w/w), at least about 98% (w/w), or at least about 99% (w/w) of the composition.

13. (canceled)

14. A method according to claim 2, wherein the composition further comprises at least one of a diluent, carrier, or auxiliary agent.

15. (canceled)

16. A method according to claim 14, wherein the phosvitin or peptide fragments thereof together with the diluent, carrier, or auxiliary agent constitute at least about 95% (w/w), at least about 96% (w/w), at least about 97% (w/w), at least about 98% (w/w), or at least about 99% (w/w) of the composition, and the composition comprises no more than about 148 ppm calcium ions (Ca²⁺), and no more than about 3.2 ppm metallic mineral ions chelated thereto.

17. A method according to claim 16, wherein in an aspect, the metallic mineral ions are iron ions (Fe²⁺), zinc ions (Zn²⁺) and manganese ions (Mn²⁺).

18. A method according to claim 2, wherein the composition exhibits antimicrobial properties.

19. A method according to claim 2, wherein the composition exhibits anti-oxidant properties.

20. A method according to claim 2, wherein the phosvitin is a protein comprising SEQ.ID NO: 1, or an amino acid sequence that is at least 80% identical thereto.

21. A method according to claim 2, wherein the phosvitin peptide fragments have a polypeptide sequence selected from the group consisting of SEQ.ID NO: 3; SEQ.ID NO: 4; SEQ.ID NO: 5; SEQ.ID NO: 6; SEQ.ID NO: 7; SEQ.ID NO: 8; SEQ.ID NO: 9; SEQ.ID NO: 10; SEQ.ID NO: 11; SEQ.ID NO: 12; SEQ.ID NO: 13; and SEQ.ID NO: 14, or a sequence that is at least 80% identical thereto.

22. A method according to claim 2, wherein the phosvitin or peptide fragments thereof are substantially free from egg proteins naturally associated therewith.

23. A product comprising:
(i) an effective preservative amount of a composition comprising phosvitin or peptide fragments thereof, wherein the phosvitin or peptide fragments thereof are substantially free of mineral ions chelated thereto; and
(ii) additional ingredients suitable for formulating the product.

24. A product according to claim 23, wherein the product is a food product, personal care product or pharmaceutical product.

25. A product according to claim 23, wherein the phosvitin or peptide fragments thereof are substantially free of bivalent mineral ions chelated thereto.

26. A product according to claim 23, wherein the phosvitin or peptide fragments thereof are substantially free of bivalent mineral ions chelated thereto, the mineral ions selected from the group consisting of calcium ions (Ca²⁺), iron ions (Fe²⁺), zinc ions (Zn²⁺), and manganese ions (Mn²⁺), wherein the phosvitin or peptide fragments thereof comprise less than about 148 ppm calcium ions (Ca²⁺), less than about 3.2 ppm iron ions (Fe²⁺), less than 2.3 ppm zinc ions (Zn²⁺), and less than about 0.033 ppm manganese ions (Mn²⁺) chelated thereto.

27-45. (canceled)

46. A preservative formulation comprising phosvitin or peptide fragments thereof substantially free of mineral ions chelated thereto, together with a diluent, a carrier, or auxiliary agent, the mineral ions selected from the group consisting of calcium ions (Ca²⁺), iron ions (Fe²⁺), zinc ions (Zn²⁺), and manganese ions (Mn²⁺), wherein the phosvitin or peptide fragments thereof comprise less than about 148 ppm calcium ions (Ca²⁺), less than about 3.2 ppm iron ions (Fe²⁺), less than 2.3 ppm zinc ions (Zn²⁺), and less than about 0.033 ppm manganese ions (Mn²⁺) chelated thereto.


SEQUENCE LISTING (14 sequences)

SEQ ID NO: 1 - protein, 217 aa, Gallus gallus
AEFGTEPDAKTSSSSSSASSTATSSSSSSASSPNRKKPMDEEENDQVKQARNKDASSSSRSSKSSNSSKRSSSKSSNSSKRSSSSSSSSSSSSRSSSSSSSSSSNSKSSSSSSKSSSSSSRSRSSSKSSSSSSSSSSSSSSKSSSSRSSSSSSKSSSHHSHSHHSGHLNGSSSSSSSSRSVSHHSHEHHSGHLEDDSSSSSSSSVLSKIWGRHEIYQ

SEQ ID NO: 2 - DNA, 650 bp, Gallus gallus
gcagagtttgggacagagcctgatgcaaaaacttcctccagctcatcttctgcctcctcaactgccacctcttcttcctcatcatctgcctcctctcctaatcgtaaaaagcctatggatgaagaggagaatgatcaagtaaagcaagcaagaaacaaagatgcaagcagcagcagcaggagcagcaagagcagtaacagcagcaagagaagcagcagcaagagcagtaacagcagcaagagaagcagcagcagcagtagcagtagcagtagcagcagcaggagcagcagcagtagcagcagtagtagcagtaacagcaagagcagcagtagcagcagcaagagcagcagtagcagcagcaggagcagaagcagcagcaagagcagcagtagcagcagcagcagtagcagcagcagcagcagcaaaagtagcagtagtaggagcagtagcagcagcagcaagtcaagcagtcaccatagccatagccatcattcagggcatctaaatggcagcagcagcagcagcagcagcagcaggtcagtgagtcaccacagccatgagcatcactcaggacatctggaagatgacagcagtagcagcagcagcagcagcgtgctttccaaaatatgggtaactcatatttgtcagta

SEQ ID NO: 3 - protein, 113 aa, Gallus gallus
AEFGTEPDAKTSSSSSSASSTATSSSSSSASSPNRKKPMDEEENDQVKQARNKDASSSSRSSKSSNSSKRSSSKSSNSSKRSSSSSSSSSSSSRSSSSSSSSSSNSKSSSSSS

SEQ ID NO: 4 - protein, 104 aa, Gallus gallus
KSSSSSSRSRSSSKSSSSSSSSSSSSSSKSSSSRSSSSSSKSSSHHSHSHHSGHLNGSSSSSSSSRSVSHHSHEHHSGHLEDDSSSSSSSSVLSKIWGRHEIYQ

SEQ ID NO: 5 - protein, 55 aa, Gallus gallus
AEFGTEPDAKTSSSSSSASSTATSSSSSSASSPNRKKPMDEEENDQVKQARNKDA

SEQ ID NO: 6 - protein, 58 aa, Gallus gallus
SSSSRSSKSSNSSKRSSSKSSNSSKRSSSSSSSSSSSSRSSSSSSSSSSNSKSSSSSS

SEQ ID NO: 7 - protein, 53 aa, Gallus gallus
KSSSSSSRSRSSSKSSSSSSSSSSSSSSKSSSSRSSSSSSKSSSHHSHSHHSG

SEQ ID NO: 8 - protein, 50 aa, Gallus gallus
LNGSSSSSSSSRSVSHHSHEHHSGHLEDDSSSSSSSSVLSKIWGRHEIYQ

SEQ ID NO: 9 - protein, 32 aa, Gallus gallus
AEFGTEPDAKTSSSSSSASSTATSSSSSSASS

SEQ ID NO: 10 - protein, 34 aa, Gallus gallus
PNRKKPMDEEENDQVKQARNKDASSSSRSSKSSN

SEQ ID NO: 11 - protein, 38 aa, Gallus gallus
SSKRSSSKSSNSSKRSSSSSSSSSSSSRSSSSSSSSSS

SEQ ID NO: 12 - protein, 43 aa, Gallus gallus
NSKSSSSSSKSSSSSSRSRSSSKSSSSSSSSSSSSSSKSSSSR

SEQ ID NO: 13 - protein, 31 aa, Gallus gallus
SSSSSSKSSSHHSHSHHSGHLNGSSSSSSSS

SEQ ID NO: 14 - protein, 39 aa, Gallus gallus
RSVSHHSHEHHSGHLEDDSSSSSSSSVLSKIWGRHEIYQ